(19) United States
(12) Patent Application Publication
(10) Pub. No.: US 2021/0338477 A1
(43) Pub. Date: Nov. 4, 2021

(54) PREPARATION METHOD AND PREPARATION APPARATUS OF HEATING FLUID USED FOR BIOLOGICAL TARGETED HYPERTHERMIA AND HYPERTHERMIA APPARATUS

(71) Applicant: Yinghui Sun, Shanghai (CN)

(72) Inventor: Yinghui Sun, Shanghai (CN)

(21) Appl. No.: 16/932,820

(22) Filed: Jul. 19, 2020

(30) Foreign Application Priority Data
Apr. 29, 2020 (CN) 202010359017.1

(51) Int. Cl. A61F 7/12; A61N 5/02; C07K 16/30; A61K 41/00

(52) U.S. Cl. CPC A61F 7/12; A61N 5/025; A61F 2007/0095; A61K 41/0052; C07K 16/30

(57) ABSTRACT

This application provides a preparation method of a heating fluid used for biological targeted hyperthermia, a heating fluid preparation device, a biological targeted hyperthermia apparatus, and a control method of the biological targeted hyperthermia, in which the biological targeted hyperthermia apparatus includes: a heating fluid injection device configured to inject a heating fluid into a body circulatory system of a therapy subject; a microwave radiation device configured to provide microwave radiation with a set wavelength and a set frequency; a microwave imaging temperature-measuring device configured to detect a temperature and a position of the heating fluid in the therapy subject, and output a thermal imaging signal; and a control device used for receiving the thermal imaging signal and controlling the microwave radiation device to output the microwave radiation with the set wavelength and the set frequency.

### CROSS REFERENCE TO RELATED APPLICATIONS

This application claims the priority to Chinese Patent Application No. 202010359017.1 filed on Apr. 29, 2020, the content of which is incorporated herein in its entirety by reference.

### TECHNICAL FIELD

This application relates to the technical field of medical equipment, and more particularly, to a preparation method and preparation apparatus of a heating fluid used for biological targeted hyperthermia in tumor therapy and a hyperthermia apparatus.

### BACKGROUND

In the current technologies, radiotherapy, chemotherapy, and surgical therapy are the most commonly used therapy methods for killing tumor cells or tumor tissue in human or animal bodies. Radiotherapy, also called radiation therapy, is a method in which a high-energy particle ray generated by a radioactive element is used to bombard tumor cells, thereby achieving the killing of tumor cells. Surgical therapy is a method in which tumor cells are removed from the human body by using a means of medical physics. For metastatic cancers and multiple tumors, the commonly used method at present is to use targeted drugs, that is chemotherapy, for delivering drugs that act on tumor cells to tumor tissue, thereby achieving the killing of the tumor tissue.

In the therapy methods mentioned above, normal tissue and cells in a human body will also be damaged during the process of killing tumor tissue. For example, in the current radiotherapy, the delivery of the drug is not very accurate, and normal tissue near the tumor tissue will also be killed, which causes damage to the patient and produce obvious side effects.

### BRIEF SUMMARY

Regarding problems that normal tissue and cells in a human body are easy to be damaged during the process of killing the tumor tissue in an actual application, which causes damage to the patient and uncomplete killing of the tumor tissue and tumor cells, at one aspect, a preparation method of a heating fluid used for biological targeted hyperthermia used in tumor therapy is provided, which is capable of preparing a heating fluid with the effect of targeted delivery so as to deliver a heating substance which generates heat in a microwave radiation field to tumor tissue. At a second aspect, a heating fluid preparation device used for biological targeted hyperthermia used in tumor therapy is provided. Based on the above-mentioned heating fluid, a biological targeted hyperthermia apparatus used for tumor therapy is provided, which can accurately identify tumor tissue or tumor cells by using the antibody naturally present in a human or animal body as a carrier and release a heating substance into the identified area, so as to accurately heat the tumor tissue or tumor cells by microwave heating, thereby killing the tumor tissue or stopping the spreading of tumor tissue or tumor cells. Based on the above-mentioned hyperthermia device, a control method of the biological targeted hyperthermia used for tumor therapy is provided, which improves the effect of killing the tumor tissue in combination with the hyperthermia device mentioned above. Particular embodiments are explained as follow.

A preparation method of a heating fluid used for biological targeted hyperthermia used in tumor therapy includes the following steps:

analyzing a medical characteristic of tumor tissue and obtaining an antibody from a therapy subject;

expanding the obtained antibody by in vitro expansion technology;

analyzing a characteristic of the antibody and preparing a heating substance capable of being combined with the antibody and generating heat in a set microwave radiation field; and

combining the heating substance with the antibody in a preparation environment to prepare a heating fluid with a set fluidity.

In the above-mentioned technical solution, by using the antibody against tumor tissue or tumor cells naturally generated in a therapy subject as a guide carrier for the targeted therapy, the heating substance can be accurately carried to tumor tissue or tumor cells to be killed. Then microwave can be used to radiate the therapy subject, so as to heat and in turn kill tumor tissue and tumor cells by using the heat generated by the heating fluid, thereby achieving accurate and non-invasive tumor therapy.

Based on the preparation method mentioned above, this application further provides a heating fluid preparation apparatus used for biological targeted hyperthermia used in tumor therapy, including:

a device configured to acquire an antibody of a therapy subject;

a device configured to purify and expand the antibody;

a device configured to prepare a specific heating substance according to a characteristic of the antibody; and

a device configured to combine the antibody and the heating substance to form a heating fluid with a set fluidity.

Based on the above-mentioned heating fluid, this application further provides a biological targeted hyperthermia apparatus used for tumor therapy, including:

a heating fluid injection device configured to inject a heating fluid into a body circulatory system of a therapy subject;

a microwave radiation device configured to provide a microwave radiation with a set wavelength and a set frequency;

a microwave imaging temperature-measuring device configured to detect a temperature and a position of the heating fluid in the therapy subject, and output a thermal imaging signal;

and a control device configured to be in signal connection with the microwave imaging temperature-measuring device and in control connection with the microwave radiation device, and used for receiving the thermal imaging signal and controlling the microwave radiation device to output the microwave radiation with a set wavelength and a set frequency.

In the above-mentioned technical solution, the heating fluid that can generate heat in the microwave radiation field is injected into the body circulation system of the therapy subject through which the heating fluid is delivered to tumor tissue or tumor cells and combined with them. Then heating is performed by microwave, the temperature of the heating area, that is, the area where the heating fluid gathers, is monitored by using the above-mentioned microwave imaging temperature-measuring device, and the wavelength and frequency of the microwave output by the microwave radiation device are adjusted in real time, thereby achieving accurate closed-loop control for microwave heating, which can kill tumor tissue or tumor cells more accurately.

Further, the biological targeted hyperthermia apparatus includes a position adjustment device configured to adjust a position of a body of the therapy subject in the microwave radiation field.

In the above-mentioned technical solution, the microwave radiation received by the therapy subject may be adjusted by adjusting the position of the body of the therapy subject in the microwave radiation field, so that the above-mentioned microwave radiation is mainly concentrated on the area where the heating fluid is located, that is, the position of tumor tissue or tumor cells.

Further, the position adjustment device includes:

a bed body, disposed in the microwave radiation field and configured to provide stable support for the therapy subject; and

a bed body driving member, configured to be connected with the control device, for changing a position of the bed body in a three-dimensional space.

Further, the biological targeted hyperthermia apparatus includes a hyperthermia solution generating device, including:

a tumor tissue imaging acquiring unit, configured to automatically collect or receive information input from outside to generate a shape, a size, and a position coordinate of tumor tissue in the therapy subject;

a heating fluid amount generating unit, configured to be in signal connection with the tumor tissue imaging acquiring unit and used to generate and output information on an amount of the heating fluid on the basis of a set algorithm according to the shape, the size and the position coordinate of the tumor tissue; and

a microwave radiation parameter generating unit, configured to be in signal connection with the tumor tissue imaging acquiring unit and used to generate and output information on a parameter of the microwave radiation according to the shape, the size, the position coordinate of the tumor tissue, and the information on the amount of the heating fluid.

In the above-mentioned technical solution, the amount of the heating fluid and the corresponding parameter of the microwave radiation can be automatically calculated according to the shape, size, and position coordinate of the tumor tissue in the therapy object, thus the tumor tissue can be accurately killed in a short time.

Further, the biological targeted hyperthermia apparatus includes a heating fluid flow direction monitoring unit configured to be in signal connection with the microwave imaging temperature-measuring device, receive the thermal imaging signal of the heating fluid, and generate and output information on a flow path and a gathering area of the heating fluid.

In the above-mentioned technical solution, on one hand, the flow direction of the heating fluid can be monitored, ensuring that the heating substance in the heating fluid can reach the tumor tissue, thus normal tissue and cells of the therapy subject can be prevented from being damaged during hyperthermia; and, on the other hand, the shape and size of the tumor tissue can be checked again by the imagings acquired after the heating fluid gathers up so as to ensure the heating fluid is sufficient, thereby ensuring the effect of the hyperthermia.

Further, the biological targeted hyperthermia apparatus includes:

a safety monitoring device, configured to be in signal connection with the microwave imaging temperature-measuring device, receive the thermal imaging signal of the heating fluid, analyze the temperature of the heating fluid and compare it with a preset value, and output a warning signal to the control device if the temperature of the heating fluid exceeds a set range; and

a safety executing device, disposed in the control device and configured to receive the warning signal and output an internal control signal for controlling the control device so as to adjust output of the microwave radiation device.

In the above-mentioned technical solution, changes in the temperature of the heating fluid and cells in the normal tissue of the therapy subject in the process of hyperthermia can be real-time monitored, so that the wavelength of the microwave or the power of the microwave radiation device can be adjusted in time when changes in the temperature exceed a preset range, so as to avoid the damage of the heating fluid or microwave on the normal tissue of the therapy subject.

Further, the microwave radiation device includes a plurality of microwave radiators disposed on a mounting frame, and a plurality of microwave radiators are slidably disposed on the mounting frame and are connected with an external microwave source and a power regulating module.

In the above-mentioned technical solution, it can not only provide a microwave radiation field with microwave radiated toward diverse directions, but also adjust the density of the microwave radiation in the microwave radiation field by separating or concentrating a plurality of microwave radiators.

Based on the above-mentioned hyperthermia device, this application further provides a control method of the biological targeted hyperthermia used for tumor therapy, including:

preparing a heating fluid with a set amount according to a shape and a size of tumor tissue in a therapy subject;

injecting the heating fluid into a body circulatory system of the therapy subject and maintaining for a set time, so as to ensure that a heating substance in the heating fluid gathers at the tumor tissue or tumor cells;

placing the therapy subject in a microwave radiation field with a set radiation intensity, monitoring and gradually adjusting the radiation intensity, so that a temperature of the heating fluid is maintained at a set value for a set time; and

separating the therapy subject from the microwave radiation field, ending the therapy.

In the above-mentioned technical solution, tumor tissue can be accurately identified and heating substance can be delivered to tumor tissue by using the antibody of the therapy subject in the heating fluid. The heating substance is heated by the microwave radiation in the microwave radiation field, so as to heat the above-mentioned tumor tissue to a set temperature for a set time, thus the tumor tissue and tumor cells are finally killed. The whole process is non-invasive so that it does not damage the normal tissue and cells in the therapy subject.

Compared with the prior art, this application has the following beneficial effects.

(1) By combining the heating substance which can generate heat in the microwave radiation field with the antibody against the tumor tissue, tumor tissue or tumor cells are accurately identified by the antibody, and tumor tissue are killed by the heat generated by the heating substance in the microwave radiation field, thus cells in normal tissue will not be damaged during the whole therapy.

(2) During the therapy, the heating fluid injected into the therapy subject will not affect the tissue and cells, and will be discharged out of the therapy subject by metabolism after the hyperthermia. This is a purely non-invasive physical therapy which does not have side effects;

(3) In the therapy method of this application, not only tumor tissue or tumor cells can be killed, but also the spread of tumor cells can be stopped to some extent, so as to prevent the worsening of the condition.

### BRIEF DESCRIPTION OF DRAWINGS

FIG. 1 is a schematic view of a preparation method of a heating fluid used for biological targeted hyperthermia;

FIG. 2 is a schematic view of a control method of biological targeted hyperthermia used for tumor therapy;

FIG. 3 is a framework schematic view of functional modules of this application; and

FIG. 4 is a schematic view of an overall structure of this application.

In the drawings:

1. heating fluid injection device;
2. microwave radiation device
3. microwave imaging temperature-measuring device
4. control device
5. position adjustment device
6. bed body
7. bed body driving member
8. hyperthermia solution generating device
9. tumor tissue imaging acquiring unit
10. heating fluid amount generating unit
11. microwave radiation parameter generating unit
12. heating fluid flow direction monitoring unit
13. safety monitoring device
14. safety executing device
15. microwave radiator
16. mounting frame

### DETAILED DESCRIPTION

This application will be further explained in detail below in combination with embodiments and figures. These examples are only an explanation of this application, and do not limit the protection scope of this application.

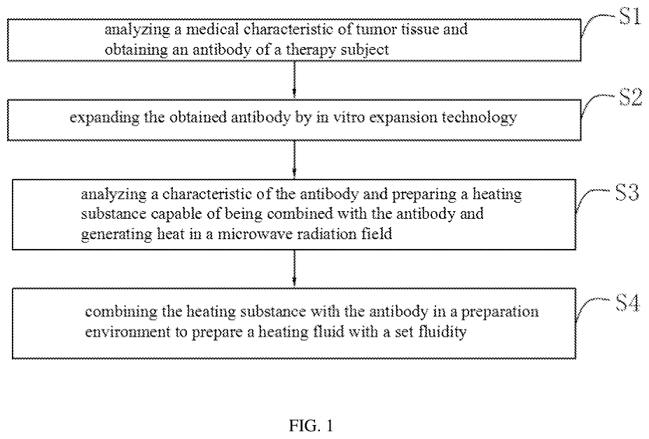

As shown in FIG. 1, a preparation method of heating fluid used for biological targeted hyperthermia used in tumor therapy includes the following steps:

S1, analyzing a medical characteristic of tumor tissue and obtaining an antibody from a therapy subject;

S2, expanding the obtained antibody by in vitro expansion technology;

S3, analyzing a characteristic of the antibody and preparing a heating substance capable of being combined with the antibody and heating in a microwave radiation field; and

S4, combining the heating substance with the antibody in a specific preparation environment to prepare a heating fluid with a set fluidity.

In the above step S1, analyzing the medical characteristic of the tumor tissue is mainly used for obtaining a corresponding antibody. Regarding different tumor issues, there are already relatively established antibody collecting methods in the current medical field, in which the antibody is an immune protein that has an inhibitory effect against the expansion of tumor tissue and tumor cells.

In the above step S2, the in vitro antibody expansion technology is adopted, in which antibody cells are placed in a specific culture dish with a suitable environment, thereby achieving in vitro cell expansion.

In the above step S3, a non-toxic substance, such as Fe₃O₄ particles or a composition thereof, is preferably adopted as the above-mentioned heating substance.

In the above step S4, for the purpose of combining the above-mentioned heating substance with the antibody, the particle size of the above-mentioned heating substance is of a nano level. The heating fluid is formed by combining a mixture of the heating substance and the antibody together with a set solution, such as physiological saline, etc., and is finally injected into the therapy subject by injection.

In the above-mentioned technical solution, by using the antibody against tumor tissue or tumor cells naturally generated in a therapy subject as a guide carrier for the targeted therapy, the heating substance can be accurately carried to tumor tissue or tumor cells to be killed. Then the therapy subject is radiated by microwave, so that the heat generated by the heating fluid is used to heat and in turn kill tumor tissue and tumor cells, thereby achieving accurate and non-invasive tumor therapy.

Based on the preparation method mentioned above, this application further provides a heating fluid preparation apparatus used for biological targeted hyperthermia used in tumor therapy, including:

a device configured to acquire an antibody of a therapy subject;

a device configured to purify and expand the antibody;

a device configured to prepare a specific heating substance according to a characteristic of the antibody; and

a device configured to combine the antibody and the heating substance to form a heating fluid with a set fluidity.

The device configured to obtain the antibody of the therapy subject may include cell separation and purification equipment by which the antibody is separated and purified from the blood of the patient. The expansion device may include a constant temperature incubator used for expanding the antibody in a culture environment with a set pH. The process and devices mentioned above may be those currently used in various laboratories, and thus the details of will not be explained hereinafter.

The device configured to prepare a specific heating substance may include a Fe₃O₄ particle preparation machine, by which Fe₃O₄ particle with a set size is prepared under a specific condition. The device configured to combine the antibody with the heating substance may be the cell expansion device itself, and a solution containing Fe₃O₄ particles with a set concentration is directly added to the culture dish as the culture medium in the process of antibody expansion.

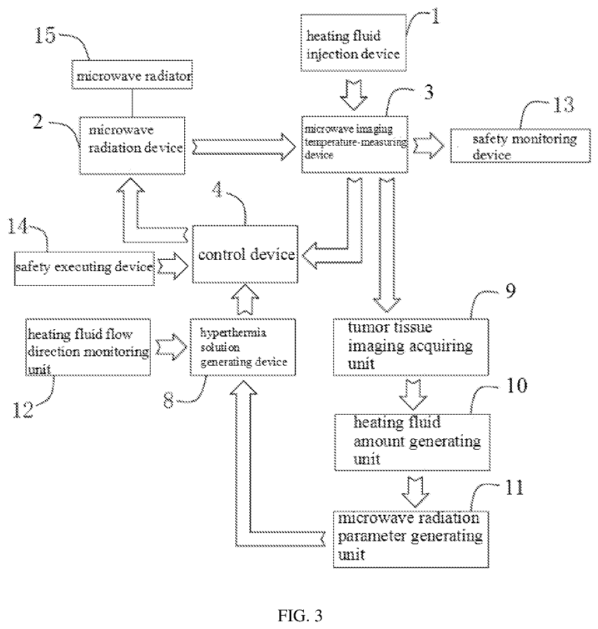

Based on the above-mentioned heating fluid, this application further includes a biological targeted hyperthermia apparatus used for tumor therapy mainly including a heating fluid injection device 1, a microwave radiation device 2, a microwave imaging temperature-measuring device 3, and a control device 4 as shown in FIG. 3.

The heating fluid injection device 1, such as a syringe or its alternative medical device, is configured to inject the heating fluid into the body circulatory system of the therapy subject. In order to reduce the time for circulation, a syringe with a long needle can be adopted as the above-mentioned heating fluid injection device 1. By using ultrasonic imaging, the heating fluid is accurately injected into the tumor tissue or tumor cells in the therapy subject, so that Fe₃O₄ particles in the heating fluid can be accurately and quickly attached to the tumor tissue.

The microwave radiation received by the therapy subject can be adjusted by adjusting the position of the body of the therapy subject in the microwave radiation field. The biological targeted hyperthermia apparatus further includes a position adjustment device 5 configured to adjust the position of the body of the therapy subject in the microwave radiation field, so that the above-mentioned microwave radiation is mainly concentrated in the area where the heating fluid is located, that is, the position of tumor tissue or tumor cells.

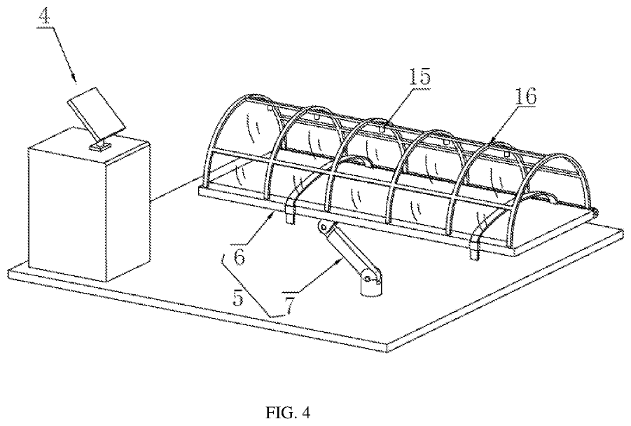

Specifically, as shown in FIG. 3 and FIG. 4, the position adjustment device 5 includes a bed body 6 and a bed body driving member 7.

The bed body 6 is disposed in the microwave radiation field and configured to provide stable support for the therapy subject. The above-mentioned bed body 6 is made of ceramic or glass so as to not block the microwave radiation. The bed body 6 is provided with an auxiliary fixing device such as a bandage, which is used to fix the therapy subject together with the bed body 6 during the hyperthermia. The bed body driving member 7 is configured to be connected with the control device 4 and used for changing the position of the bed body 6 in a three-dimensional space. In one embodiment, as shown in FIG. 4, the bed body may be implemented as two connected mechanical arms, and the controllers of the mechanical arms are in control connection with the control device 4 so as to drive the bed body 6 to move in the three-dimensional space.

The microwave radiation device 2 is configured to provide a microwave radiation field with a set wavelength and a set frequency. In one embodiment, the above-mentioned microwave radiation device 2 includes a plurality of microwave radiators 15 disposed on a mounting frame 16, and a plurality of microwave radiators 15 are slidably disposed on the mounting frame 16 and are connected with an external microwave source and a power regulating module. Preferably, the mounting frame 16 are arranged in a ring shape or tile shape, and the microwave radiators 15 are detachably disposed on the mounting frame 16 through a clamping member. In the above-mentioned technical solution, it can not only provide a microwave radiation field with microwave radiated toward diverse directions, but also adjust the density of the microwave radiation in the microwave radiation field by separating or concentrating a plurality of microwave radiators.

The microwave imaging temperature-measuring device 3 is configured to detect the temperature and the position of the heating fluid in the therapy subject, and output a thermal imaging signal. Combining a microwave thermometric indicator with a microwave imager is adopted as the microwave imaging temperature-measuring device 3. It should be noted that the imaging of the tumor tissue may be obtained from other medical devices such as a CT machine before hyperthermia.

In this application, the control device 4, configured to be in signal connection with the microwave imaging temperature-measuring device 3 and in control connection with the microwave radiation device 2, is used for receiving the thermal imaging signal and controlling the microwave radiation device 2 to output microwave radiation with a set wavelength and a set frequency. In one embodiment, a computer or a microprocessor loaded with a set algorithm program may be adopted as the above-mentioned control device 4.

In order to kill the tumor tissue more accurately, the biological targeted hyperthermia apparatus further includes a hyperthermia solution generating device 8, including a tumor tissue imaging acquiring unit 9, a heating fluid amount generating unit 10, and a microwave radiation parameter generating unit 11.

The tumor tissue imaging acquiring unit 9 is configured to automatically collect or receive information input from outside to generate a shape, a size, and a position coordinate of tumor tissue in the therapy subject. The above-mentioned information on the shape and size of the tumor tissue can be obtained by a way in which microwave is generated by the microwave radiation device 2 and then reflected or dissipated by the body of the therapy subject, and the shape and size of the tumor tissue can be displayed on the microwave imaging temperature-measuring device 3, or by a way in which CT scanning is used before hyperthermia. The heating fluid amount generating unit 10, configured to be in signal connection with the tumor tissue imaging acquiring unit 9, is used to generate and output information on an amount of the heating fluid on the basis of a set algorithm according to the shape, the size and the position coordinate of the tumor tissue. The microwave radiation parameter generating unit 11, configured to be in signal connection with the tumor tissue imaging acquiring unit 9, is used to generate and output information on a parameter of the microwave radiation according to the shape, the size, the position coordinate of the tumor tissue, and the information on the amount of the heating fluid. The information on the parameter of the microwave radiation includes but is not limited to microwave wavelength, frequency, radiation duration, and other parameters.

In the above-mentioned technical solution, the amount of the heating fluid and the corresponding parameter of the microwave radiation can be automatically calculated according to the shape, size, and position coordinate of the tumor tissue in the therapy object, thus the tumor tissue can be accurately killed in a short time.

Further, the biological targeted hyperthermia apparatus further includes a heating fluid flow direction monitoring unit 12 configured to be in signal connection with the microwave imaging temperature-measuring device 3, receive the thermal imaging signal of the heating fluid, and generate and output information on a flow path and a gathering area of the heating fluid. The above-mentioned heating fluid flow direction monitoring unit 12 includes a program module disposed inside the control device 4 which periodically collects the signal output by the microwave imaging temperature-measuring device 3 and records information on the position of the heating fluid at different time points. When the heating fluid stably gathers at a certain position of the therapy subject, it can be confirmed that the heating fluid has been stably attached onto the tumor tissue or tumor cells. In the above-mentioned technical solution, on one hand, the flow direction of the heating fluid can be monitored, ensuring that the heating substance in the heating fluid can reach the tumor tissue, thus normal tissue and cells of the therapy subject can be prevented from being damaged during hyperthermia; on the other hand, the shape and size of the tumor tissue can be checked again by the imaging obtained after the heating fluid gathers up so as to ensure the heating fluid is sufficient, thereby ensuring the effect of the hyperthermia.

The biological targeted hyperthermia apparatus further includes a safety monitoring device 13 and a safety executing device 14.

The safety monitoring device 13 is configured to be in signal connection with the microwave imaging temperature-measuring device 3, receive the thermal imaging signal of the heating fluid, analyze the temperature of the heating fluid and compare it with a preset value, and output a warning signal to the control device 4 if the temperature of the heating fluid exceeds a preset range. The safety executing device 14, disposed in the control device 4, is configured to receive the warning signal and output an internal control signal for controlling the control device so as to adjust output of the microwave radiation device 2. In the above-mentioned technical solution, changes in the temperature of the heating fluid and cells in the normal tissue of the therapy subject in the process of hyperthermia can be real-time monitored, so that the wavelength of the microwave or the power of the microwave radiation device 2 can be adjusted in time when changes in the temperature exceed a preset range, so as to avoid the damage of the heating fluid or microwave on the normal tissue of the therapy subject. In one embodiment, both the safety monitoring device 13 and the safety executing device 14 can be implemented by disposing the program module inside the control device 4.

The working principle and beneficial effects of the biological targeted hyperthermia apparatus in this application are as follow.

The heating fluid that can generate heat in the microwave radiation field is injected into the body circulation system of the therapy subject through which the heating fluid is delivered to tumor tissue or tumor cells and combined with them. Then heating is performed by microwave, the temperature of the heating area, that is, the area where the heating fluid gathers, is monitored by using the above-mentioned microwave imaging temperature-measuring device 3, and the wavelength and frequency of the microwave output by the microwave radiation device 2 are adjusted in real time, thereby achieving accurate closed-loop control of microwave heating, which can kill tumor tissue or tumor cells more accurately.

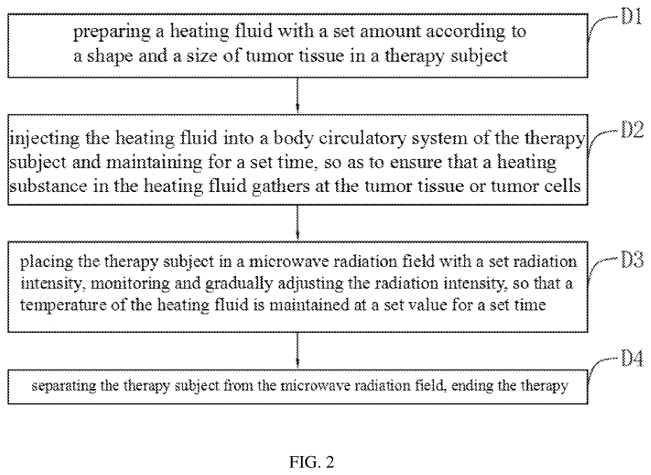

Based on the above-mentioned hyperthermia device, this application also provides a control method of the biological targeted hyperthermia used for tumor therapy, as shown in FIG. 2, including:

D1, preparing a heating fluid with a set amount according to a shape and a size of tumor tissue in a therapy subject;

D2, injecting the heating fluid into a body circulatory system of the therapy subject and maintaining for a set time, so as to ensure that a heating substance in the heating fluid gathers at the tumor tissue or tumor cells;

D3, placing the therapy subject in a microwave radiation field with a set radiation intensity, monitoring and gradually adjusting the radiation intensity, so that a temperature of the heating fluid is maintained at a set value for a set time; and

D4, separating the therapy subject from the microwave radiation field, ending the therapy.

By repeating the hyperthermia, tumor tissue can be accurately identified and heating substance can be delivered to tumor tissue by using the antibody of the therapy subject in the heating fluid. The heating substance is heated by the microwave radiation in the microwave radiation field, so as to heat the above-mentioned tumor tissue to a set temperature for a set time, thus the tumor tissue and tumor cells are finally killed. The whole process is non-invasive so that it does not damage the normal tissue and cells in the therapy subject.

The embodiments described in the detailed description are all preferred embodiments of this application, and should not be considered as limit the scope of this application thereto. Therefore, all the equivalent changes made to the structure, shape and principle of this application should be encompassed within the protection scope of this application.

What is claimed is:

1. A preparation method of a heating fluid used for biological targeted hyperthermia used in tumor therapy comprising the following steps:
analyzing a medical characteristic of tumor tissue and obtaining an antibody of a therapy subject;
expanding the obtained antibody by in vitro expansion technology;
analyzing a characteristic of the antibody and preparing a heating substance capable of being combined with the antibody and generating heat in a microwave radiation field; and
combining the heating substance with the antibody in a preparation environment to prepare a heating fluid with a set fluidity.

2. A heating fluid preparation device used for biological targeted hyperthermia used in tumor therapy, comprising:
a device configured to acquire an antibody of a therapy subject;
a device configured to purify and expand the antibody;
a device configured to prepare a specific heating substance according to a characteristic of the antibody; and
a device configured to combine the antibody and the heating substance to form a heating fluid with a set fluidity.

3. A biological targeted hyperthermia apparatus used for tumor therapy, comprising:
a heating fluid injection device configured to inject a heating fluid into a body circulatory system of a therapy subject;
a microwave radiation device configured to provide a microwave radiation field with a set wavelength and a set frequency;
a microwave imaging temperature-measuring device configured to detect a temperature and a position of the heating fluid in the therapy subject, and output a thermal imaging signal; and
a control device configured to be in signal connection with the microwave imaging temperature-measuring device and in control connection with the microwave radiation device, and used for receiving the thermal imaging signal and controlling the microwave radiation device to output the microwave radiation with the set wavelength and the set frequency.

4. The biological targeted hyperthermia apparatus used for tumor therapy according to claim 3, further comprising a position adjustment device configured to adjust a position of a body of the therapy subject in the microwave radiation field.

5. The biological targeted hyperthermia apparatus used for tumor therapy according to claim 4, further comprising:
a bed body disposed in the microwave radiation field and configured to provide a stable support for the therapy subject; and
a bed body driving member configured to be connected with the control device, for changing a position of the bed body in a three-dimensional space.

6. The biological targeted hyperthermia apparatus used for tumor therapy according to claim 3, further comprising a hyperthermia solution generating device, comprising:
a tumor tissue imaging acquiring unit, configured to automatically collect or receive information input from outside and generate a shape, a size, and a position coordinate of tumor tissue in the therapy subject;
a heating fluid amount generating unit, configured to be in signal connection with the tumor tissue imaging acquiring unit and used to generate and output information on an amount of the heating fluid on the basis of a set algorithm according to the shape, the size and the position coordinate of the tumor tissue; and
a microwave radiation parameter generating unit, configured to be in signal connection with the tumor tissue imaging acquiring unit and used to generate and output information on a parameter of the microwave radiation according to the shape, the size, the position coordinate of the tumor tissue, and the information on the amount of the heating fluid.

7. The biological targeted hyperthermia apparatus used for tumor therapy according to claim 6, further comprising a heating fluid flow direction monitoring unit configured to be in signal connection with the microwave imaging temperature-measuring device, receive the thermal imaging signal of the heating fluid, and generate and output information on a flow path and a gathering area of the heating fluid.

8. The biological targeted hyperthermia apparatus used for tumor therapy according to claim 3, further comprising:
a safety monitoring device, configured to be in signal connection with the microwave imaging temperature-measuring device, receive the thermal imaging signal of the heating fluid, analyze the temperature of the heating fluid and compare it with a set value, and output a warning signal to the control device if the temperature of the heating fluid exceeds a set range; and
a safety executing device, disposed in the control device and configured to receive the warning signal and output an internal control signal for controlling the control device so as to adjust output of the microwave radiation device.

9. The biological targeted hyperthermia apparatus used for tumor therapy according to claim 3, wherein the microwave radiation device comprises a plurality of microwave radiators disposed on a mounting frame, and a plurality of microwave radiators are slidably disposed on the mounting frame and are connected with an external microwave source and a power regulating module.
